(19) United States
(12) Patent Application Publication
(10) Pub. No.: US 2024/0299361 A1
(43) Pub. Date: Sep. 12, 2024

(54) VETERINARY TRANSDERMAL GEL COMPRISING ONDANSETRON, AND RELATED METHODS

(71) Applicant: Verté Therapeutics, LLC, New York, NY (US)

(72) Inventors: Lesley Corene Rausch-Derra, Parkville, MO (US); Susan Mancini Cady, Yardley, PA (US); Rex Allen Henry, Lenexa, KS (US); Sai Sree Lekha Akkineni, Durham, NC (US); Kevin Scott Warner, South Jordan, UT (US); Ann Ruth Donoghue, Fort Collins, CO (US); Srikanth Manne, Cary, NC (US); Vijendra Nalamothu, Raleigh, NC (US)

(21) Appl. No.: 18/424,380

(22) Filed: Jan. 26, 2024

Related U.S. Application Data
(60) Provisional application No. 63/448,816, filed on Feb. 28, 2023.

(51) Int. Cl. A61K 31/4178; A61K 9/00; A61K 9/06; A61K 47/10; A61K 47/38; A61P 1/08

(52) U.S. Cl. CPC A61K 31/4178; A61K 9/0014; A61K 9/06; A61K 47/10; A61K 47/38; A61P 1/08

(57) ABSTRACT

Disclosed is a pharmaceutical composition, comprising about 2% to about 10% w/w ondansetron or a pharmaceutically acceptable salt thereof, about 10% to about 50% w/w of one or more solvents, about 10% to about 50% w/w of one or more skin penetration enhancers, about 1% to about 5% w/w of one or more preservatives, and about 1% to about 20% w/w of one or more gelling agents. Also disclosed is a method for preventing or treating nausea and/or vomiting, comprising administering (e.g., transdermally) to a subject in need thereof a therapeutically effective amount of the pharmaceutical composition, thereby preventing or treating the nausea and/or vomiting.

### RELATED APPLICATION

This application claims benefit of priority to U.S. Provisional Patent Application No. 63/448,816, filed Feb. 28, 2023.

### BACKGROUND

Ondansetron is a serotonin (5-hydroxytryptamine) subtype 3 (5-HT3) receptor antagonist used in the management of nausea and vomiting. It is used to prevent and control nausea and vomiting after cancer chemotherapy, radiotherapy, and surgery in humans. It is also used off-label to treat nausea and vomiting in cats and dogs.

Ondansetron may be given orally in the form of a tablet or liquid. However, oral administration to cats and dogs can be difficult. Oral administration is unpleasant in an already nauseous human, dog, or cat. Also, the drug can be vomited before being absorbed by the patient. Accordingly, improved formulations of ondansetron and routes of administration are needed.

### SUMMARY OF THE INVENTION

The present invention provides a pharmaceutical composition, comprising about 2% to about 10% w/w ondansetron or a pharmaceutically acceptable salt thereof, about 10% to about 50% w/w of one or more solvents, about 10% to about 50% w/w of one or more skin penetration enhancers, about 1% to about 5% w/w of one or more preservatives, and about 1% to about 20% w/w of one or more gelling agents.

Also disclosed herein is a method for preventing or treating nausea and/or vomiting, comprising administering to a subject in need thereof a therapeutically effective amount of the pharmaceutical composition of the present invention, thereby preventing or treating the nausea and/or vomiting.

### BRIEF DESCRIPTION OF THE DRAWINGS

FIG. 1A displays the solubility of Ondansetron HCl in dimethyl isosorbide, diisopropyl adipate, PEG 40-Hydrogenated castor oil, diethylene glycol monoethyl ether, or PPG15-stearylether.

FIG. 1B displays the solubility of Ondansetron HCl in isopropyl myristate, absolute alcohol, glycerin, purified water, or propylene glycol.

FIG. 1C displays the solubility of Ondansetron HCl in hexylene glycol, benzyl alcohol, castor oil, PEG 400, and DMSO.

FIG. 1D displays the solubility of Ondansetron HCl in oleic acid, polysorbate 80, medium chain triglycerides, propylene carbonate, or cocyl caprylocaprate.

FIG. 1E displays the solubility of Ondansetron HCl in isopropyl alcohol, diethyl sebacate, citrate buffer USP, oleyl alcohol, or cyclomethicone.

FIG. 1F displays the solubility of Ondansetron HCl in hexylene glycol+polysorbate 80, hexylene glycol+isopropyl myristate, hexylene glycol+cocyl caprylocaprate, hexylene glycol+MCT, or isopropyl alcohol+polysorbate 80.

FIG. 1G displays the solubility of Ondansetron HCl in isopropyl alcohol+isopropyl myristate, isopropyl alcohol+cocyl caprylocaprate, isopropyl alcohol+mct, glycerin+polysorbate 80, or Glycerin+isopropyl myristate.

FIG. 1H displays the solubility of Ondansetron HCl in purified water+polysorbate 80, purified water+isopropyl myristate, purified water+cocyl caprylocaprate, Purified water+MCT, or propylene carbonate+polysorbate 80.

FIG. 1I displays the solubility of Ondansetron HCl in propylene carbonate+isopropyl myristate, Propylene carbonate+cocyl caprylocaprate, Propylene carbonate+MCT, PEG 400+polysorbate 80, and PEG 400+isopropyl myristate.

FIG. 1J displays the solubility of Ondansetron HCl in PEG 400+cocyl caprylocaprate or PEG 400+MCT.

FIG. 1K displays the solubility of Ondansetron HCl in citrate buffer, USP pH 3.0 or PEG 400+MCT.

FIG. 2A displays mean PK data for various formulations given at 3 mg/kg in the pinna to a dog.

FIG. 2B displays on an exponential graph the same mean PK data shown in FIG. 2A.

FIG. 3A displays mean PK data for formulation 13B at various doses administered once in the pinna.

FIG. 3B displays mean PK data for formulation 13B at various doses administered every 12 h for 60 h in the pinna.

FIG. 4 displays bar graphs of the number of emesis episodes, retching episodes, and nausea time after treatment with 13B. Bar charts of least squares means by treatment groups. The error bar represents the standard error of the least squares mean.

FIG. 5A displays a bar graph of the number of vomiting episodes after treatment with 13B, over 6 hours post-cisplatin administration.

FIG. 5B displays a bar graph of the number of retching episodes after treatment with 13B, over 6 hours post-cisplatin administration.

FIG. 5C displays a bar graph of time spent showing nausea signs after treatment with 13B, over 6 hours post-cisplatin administration.

FIG. 6A displays plasma Ondansetron concentration of various dogs following transdermal administration of 13B at 18 mg/kg every 12 h for 3 doses, then 6 mg/kg every 12 h for 10 doses.

FIG. 6B displays plasma Ondansetron concentration of various dogs following transdermal administration of 13B at 18 mg/kg every 12 h for 3 doses, then every 24 h for 5 doses.

FIG. 6C displays plasma Ondansetron concentration of various dogs following transdermal administration of 13B at 18 mg/kg every 12 h for 5 doses, then every 24 h for 4 doses.

FIG. 6D displays plasma Ondansetron concentration various dogs following transdermal administration of 13B at 18 mg/kg every 12 h for 4 doses, then every 24 h for 5 doses.

FIG. 7A displays plasma Ondansetron concentration of various cats following transdermal gel administration of 13B at 1 mg/kg on each pinna.

FIG. 7B displays plasma Ondansetron concentration of various cats following transdermal gel administration of 13B at 12 mg/kg on abdomen.

### DETAILED DESCRIPTION OF THE INVENTION

The present invention provides a pharmaceutical composition, comprising about 2% to about 10% w/w ondansetron or a pharmaceutically acceptable salt thereof, about 10% to about 50% w/w of one or more solvents, about 10% to about 50% w/w of one or more skin penetration enhancers, about 1% to about 5% w/w of one or more preservatives, and about 1% to about 20% w/w of one or more gelling agents.

The composition is formulated as a transdermal gel formulation to treat nausea and/or vomiting in a subject, e.g., a human, a non-human primate, a dog or a cat.

The transdermal gel is applied to the skin and delivers the active drug across the skin. Transdermal delivery can avoid first-pass metabolism and maintain plasma drug concentration constantly. Additionally, transdermal delivery may be appropriate for special populations/subjects where oral administration is difficult.

### Compositions of the Invention

The present invention provides a pharmaceutical composition, comprising about 2% to about 10% w/w ondansetron or a pharmaceutically acceptable salt thereof, about 10% to about 50% w/w of one or more solvents, about 10% to about 50% w/w of one or more skin penetration enhancers, about 1% to about 5% w/w of one or more preservatives, and about 1% to about 20% w/w of one or more gelling agents.

In certain embodiments, the one or more solvents are selected from the group consisting of propylene glycol, hexylene glycol, isopropyl alcohol, ethanol, isopropyl myristate, dimethyl sulfoxide, and water.

In certain embodiments, the one or more solvents are selected from the group consisting of propylene glycol and hexylene glycol.

In certain embodiments, the one or more skin penetration enhancers are selected from the group consisting of diethylene glycol monoethyl ether, oleyl alcohol, oleic acid, dimethyl isosorbide, cocoyl caprylocaprate, and medium chain triglyceride.

In certain embodiments, the medium chain triglyceride comprises caproic acid, caprylic acid, capric acid, or lauric acid moieties or combinations thereof.

In certain embodiments, the one or more skin penetration enhancers are selected from the group consisting of diethylene glycol monoethyl ether and dimethyl isosorbide.

In certain embodiments, the one or more preservatives are selected from the group consisting of benzyl alcohol, sorbic acid, benzoic acid, and phenoxyethanol.

In certain embodiments, the one or more preservatives are selected from the group consisting of benzyl alcohol and phenoxyethanol.

In certain embodiments, the one or more gelling agents are selected from the group consisting of hydroxypropyl cellulose, hydroxypropylmethyl cellulose, hydroxyethyl cellulose, sodium carboxymethyl cellulose, and sodium alginate.

In certain embodiments, the one or more gelling agents are selected from the group consisting of hydroxypropyl cellulose and hydroxypropylmethyl cellulose.

In certain embodiments, the pharmaceutical composition comprises about 5% to about 10% w/w ondansetron or a pharmaceutically acceptable salt thereof.

In certain embodiments, the pharmaceutical composition comprises about 4% to about 8% w/w ondansetron or a pharmaceutically acceptable salt thereof.

In certain embodiments, the pharmaceutical composition comprises about 5% to about 6% w/w ondansetron or a pharmaceutically acceptable salt thereof.

In certain embodiments, the pharmaceutical composition comprises about 5.25% to about 5.75% w/w ondansetron or a pharmaceutically acceptable salt thereof.

In certain embodiments, the pharmaceutical composition comprises about 6% to about 7% w/w ondansetron or a pharmaceutically acceptable salt thereof.

In certain embodiments, the pharmaceutical composition comprises about 6% to about 6.5% w/w ondansetron or a pharmaceutically acceptable salt thereof.

In certain embodiments, the pharmaceutical composition comprises about 6.2% w/w ondansetron or a pharmaceutically acceptable salt thereof.

In certain embodiments, the pharmaceutical composition comprises about 5.5% w/w ondansetron or a pharmaceutically acceptable salt thereof.

In certain embodiments, the pharmaceutical composition comprises about 5.0% w/w ondansetron or a pharmaceutically acceptable salt thereof.

In certain embodiments, the pharmaceutical composition comprises about 10% to about 45% w/w of the one or more solvents.

In certain embodiments, the pharmaceutical composition comprises about 25% to about 35% w/w of the one or more solvents.

In certain embodiments, the pharmaceutical composition comprises about 28% to about 32% w/w of the one or more solvents.

In certain embodiments, the pharmaceutical composition comprises about 29.4% w/w of a first solvent.

In certain embodiments, the pharmaceutical composition comprises about 1% w/w of a second solvent.

In certain embodiments, the first solvent is ethylene glycol.

In certain embodiments, the second solvent is hexylene glycol.

In certain embodiments, the pharmaceutical composition comprising about 30% to about 50% w/w of the one or more skin penetration enhancers.

In certain embodiments, the pharmaceutical composition comprising about 35% to about 45% w/w of the one or more skin penetration enhancers.

In certain embodiments, the pharmaceutical composition comprising about 38% w/w of a first skin penetration enhancer.

In certain embodiments, the pharmaceutical composition comprising about 5% w/w of a second skin penetration enhancer.

In certain embodiments, the first skin penetration enhancer is diethylene glycol monoethyl ether.

In certain embodiments, the second skin penetration enhancer is dimethyl isosorbide.

In certain embodiments, the pharmaceutical composition comprises about 1% to about 4% w/w of the one or more preservatives.

In certain embodiments, the pharmaceutical composition comprises about 2% w/w of a first preservative.

In certain embodiments, the pharmaceutical composition comprises about 1% w/w of a second preservative.

In certain embodiments, the first preservative is diethylene benzyl alcohol.

In certain embodiments, the second preservative is phenoxyethanol.

In certain embodiments, the pharmaceutical composition comprises about 1% to about 9% w/w of the one or more gelling agents.

In certain embodiments, the pharmaceutical composition comprises about 1% to about 1.75% w/w of the one or more gelling agents.

In certain embodiments, the pharmaceutical composition comprises about 1% w/w of the one or more gelling agents.

In certain embodiments, the pharmaceutical composition comprises about 1% w/w of hydroxypropyl cellulose as the one or more gelling agents.

In certain embodiments, the pharmaceutical composition comprises:
(i) about 2% to about 10% w/w ondansetron or a pharmaceutically acceptable salt thereof;
(ii) about 20% to about 50% w/w of propylene glycol;
(iii) about 20% to about 50% w/w of one or more skin penetration enhancers selected from diethylene glycol monoethyl ether, dimethyl isosorbide, and medium chain triglyceride;
(iv) about 1% to about 3% w/w of one or more preservatives selected from benzyl alcohol and phenoxyethanol; and
(v) about 1% to about 10% w/w of one or more gelling agents selected hydroxypropyl cellulose and hydroxypropylmethyl cellulose.

In certain embodiments, the pharmaceutical composition comprises about 5% to about 10% w/w ondansetron or a pharmaceutically acceptable salt thereof.

In certain embodiments, the pharmaceutical composition comprises about 4% to about 8% w/w ondansetron or a pharmaceutically acceptable salt thereof.

In certain embodiments, the pharmaceutical composition comprises about 5% to about 6% w/w ondansetron or a pharmaceutically acceptable salt thereof.

In certain embodiments, the pharmaceutical composition comprises about 5.25% to about 5.75% w/w ondansetron or a pharmaceutically acceptable salt thereof.

In certain embodiments, the pharmaceutical composition comprises about 6% to about 7% w/w ondansetron or a pharmaceutically acceptable salt thereof.

In certain embodiments, the pharmaceutical composition comprises about 6% to about 6.5% w/w ondansetron or a pharmaceutically acceptable salt thereof.

In certain embodiments, the pharmaceutical composition comprises about 6.2% w/w ondansetron or a pharmaceutically acceptable salt thereof.

In certain embodiments, the pharmaceutical composition comprises about 5.5% w/w ondansetron or a pharmaceutically acceptable salt thereof.

In certain embodiments, the pharmaceutical composition comprises about 5.0% w/w ondansetron or a pharmaceutically acceptable salt thereof.

In certain embodiments, the pharmaceutical composition comprises about 20% to about 40% w/w propylene glycol.

In certain embodiments, the pharmaceutical composition comprises about 22% to about 38% w/w propylene glycol.

In certain embodiments, the pharmaceutical composition comprises about 35% to about 50% of the one or more skin penetration enhancers.

In certain embodiments, the pharmaceutical composition comprises about 5% to about 20% w/w of a medium chain triglyceride.

In certain embodiments, the pharmaceutical composition comprises about 5% to about 18% w/w of a medium chain triglyceride.

In certain embodiments, the pharmaceutical composition comprises about 2% to about 3% of the one or more preservatives.

In certain embodiments, the pharmaceutical composition comprises 1% to 5% of the one or more gelling agents.

In certain embodiments, the pharmaceutical composition comprises about 1% to about 3% of the one or more gelling agents.

In certain embodiments, the pharmaceutical composition comprises about 1% to about 3% hydroxypropyl cellulose as the one or more gelling agents.

In certain embodiments, the pharmaceutical composition comprises about 1% hydroxypropyl cellulose as the one or more gelling agents.

In certain embodiments, the pharmaceutical composition comprises about 3% hydroxypropyl cellulose as the one or more gelling agents.

In certain embodiments, the pharmaceutical composition comprises:
(i) about 5% to about 10% w/w ondansetron or a pharmaceutically acceptable salt thereof; (ii) about 28% to about 32% w/w of a solvent;
(iii) about 35% to about 40% w/w of a skin penetration enhancer;
(iv) about 0.05% to about 2% w/w of a preservative; and
(v) about 0.5% to about 1% w/w of a gelling agent.

In certain embodiments, the pharmaceutical composition comprises about 5 to 6% w/w ondansetron or a pharmaceutically acceptable salt thereof.

In certain embodiments, the pharmaceutical composition comprises about 5% w/w ondansetron or a pharmaceutically acceptable salt thereof.

In certain embodiments, the pharmaceutical composition comprises about 5.25% to about 5.75% w/w ondansetron or a pharmaceutically acceptable salt thereof.

In certain embodiments, the pharmaceutical composition comprises about 6% to about 7% w/w ondansetron or a pharmaceutically acceptable salt thereof.

In certain embodiments, the pharmaceutical composition comprises about 6% to about 6.5% w/w ondansetron or a pharmaceutically acceptable salt thereof.

In certain embodiments, the pharmaceutical composition comprises about 6.2% w/w ondansetron or a pharmaceutically acceptable salt thereof.

In certain embodiments, the pharmaceutical composition comprises about 5.5% w/w ondansetron or a pharmaceutically acceptable salt thereof.

In certain embodiments, the pharmaceutical composition comprises about 5.0% w/w ondansetron or a pharmaceutically acceptable salt thereof.

In certain embodiments, the pharmaceutical composition comprises about 28.5% to about 29.5% w/w of the solvent.

In certain embodiments, the pharmaceutical composition comprises about 29% to about 29.5% w/w of the solvent.

In certain embodiments, the pharmaceutical composition comprises about 29.4% w/w of the solvent.

In certain embodiments, the pharmaceutical composition comprises about 37.5% to about 38.5% w/w of the skin penetration enhancer.

In certain embodiments, the pharmaceutical composition comprises about 38% w/w of the skin penetration enhancer.

In certain embodiments, the pharmaceutical composition comprises about 1% to about 2% of the preservative.

In certain embodiments, the pharmaceutical composition comprises about 2% w/w of the preservative.

In certain embodiments, the pharmaceutical composition comprises about 1% to about 2% of w/w the gelling agent.

In certain embodiments, the pharmaceutical composition comprises about 1% w/w of the gelling agent.

In certain embodiments, the solvent is propylene glycol, the skin penetration enhancer is diethylene glycol monoethyl ether, the preservative is benzyl alcohol, and the gelling agent is hydroxypropyl cellulose.

In certain embodiments, the ondansetron or pharmaceutically acceptable salt thereof is ondansetron hydrochloride.

In certain embodiments, the ondansetron or pharmaceutically acceptable salt thereof is ondansetron hydrochloride dihydrate.

In certain embodiments, the pharmaceutical composition comprises:
(i) about 6.2% w/w ondansetron hydrochloride dihydrate;
(ii) about 29.4% w/w propylene glycol;
(iii) about 38% w/w diethylene glycol monoethyl ether;
(iv) about 2% w/w benzyl alcohol; and
(v) about 1% w/w hydroxypropyl cellulose.

In certain embodiments, the pharmaceutical composition comprises:
(i) about 6.24% w/w ondansetron hydrochloride dihydrate;
(ii) about 29.4% w/w propylene glycol;
(iii) about 38% w/w diethylene glycol monoethyl ether;
(iv) about 2% w/w benzyl alcohol; and
(v) about 1% w/w hydroxypropyl cellulose.

In certain embodiments, the pharmaceutical composition further comprising (vi) a humectant, (vi) a skin conditioning agent, and/or (vii) a solubilizer.

In certain embodiments, the pharmaceutical composition comprises about 15% to about 20% w/w of the humectant.

In certain embodiments, the pharmaceutical composition comprises about 0.1% to about 20 w/w of the skin conditioning agent.

In certain embodiments, the pharmaceutical composition comprises about 0.1% to about 20 w/w of the solubilizer.

In certain embodiments, the skin conditioning agent is an organosilicon compound.

In certain embodiments, the organosilicon compound is cyclomethicone.

In certain embodiments, the pharmaceutical composition is a topical gel.

In certain embodiments, the pharmaceutical composition is a gel (e.g., a transdermal gel).

Also disclosed is a pharmaceutical composition, comprising about 2% to about 10% w/w ondansetron or a pharmaceutically acceptable salt thereof, about 20% to about 80% w/w of one or more solvents, about 1% to about 50% w/w of one or more skin penetration enhancers, about 0.05% to about 5% w/w of one or more preservatives, and about 0.5% to about 20% w/w of one or more gelling agents.

In certain embodiments, the one or more solvents are selected from the group consisting of propylene glycol, hexylene glycol, isopropyl alcohol, ethanol, isopropyl myristate, dimethyl sulfoxide, and water.

In certain embodiments, the one or more solvents are selected from the group consisting of propylene glycol and hexylene glycol.

In certain embodiments, the one or more skin penetration enhancers are selected from the group consisting of diethylene glycol monoethyl ether, oleyl alcohol, oleic acid, dimethyl isosorbide, cocoyl caprylocaprate, and medium chain triglyceride.

In certain embodiments, the medium chain triglyceride comprises caproic acid, caprylic acid, capric acid, or lauric acid moieties or combinations thereof.

In certain embodiments, the one or more skin penetration enhancers are selected from the group consisting of diethylene glycol monoethyl ether and dimethyl isosorbide.

In certain embodiments, the one or more preservatives are selected from the group consisting of benzyl alcohol, sorbic acid, benzoic acid, and phenoxyethanol.

In certain embodiments, the one or more preservatives are selected from the group consisting of benzyl alcohol and phenoxyethanol.

In certain embodiments, the one or more gelling agents are selected from the group consisting of hydroxypropyl cellulose, hydroxypropylmethyl cellulose, hydroxyethyl cellulose, sodium carboxymethyl cellulose, and sodium alginate.

In certain embodiments, the one or more gelling agents are selected from the group consisting of hydroxypropyl cellulose and hydroxypropylmethyl cellulose.

In certain embodiments, the pharmaceutical composition comprises about 5% to about 10% w/w ondansetron or a pharmaceutically acceptable salt thereof.

In certain embodiments, the pharmaceutical composition comprises about 5% to about 6% w/w ondansetron or a pharmaceutically acceptable salt thereof.

In certain embodiments, the pharmaceutical composition comprises about 5% w/w ondansetron or a pharmaceutically acceptable salt thereof.

In certain embodiments, the pharmaceutical composition comprises about 5.5% w/w ondansetron or a pharmaceutically acceptable salt thereof.

In certain embodiments, the pharmaceutical composition comprises about 20% to about 78% w/w of the one or more solvents.

In certain embodiments, the pharmaceutical composition comprises about 20% to about 50% w/w of the one or more solvents.

In certain embodiments, the pharmaceutical composition comprises about 20% to about 45% w/w of the one or more solvents.

In certain embodiments, the pharmaceutical composition comprises about 28.5% to about 29.5% w/w of the one or more solvents.

In certain embodiments, the pharmaceutical composition comprises about 29.4% w/w of the one or more solvents.

In certain embodiments, the pharmaceutical composition comprises about 10% to about 50% w/w of the one or more skin penetration enhancers.

In certain embodiments, the pharmaceutical composition comprises about 30% to about 40% w/w of the one or more skin penetration enhancers.

In certain embodiments, the pharmaceutical composition comprises about 37.5% to about 38.5% w/w of the one or more skin penetration enhancers.

In certain embodiments, the pharmaceutical composition comprises about 38% w/w of the one or more skin penetration enhancers.

In certain embodiments, the pharmaceutical composition comprises about 1% to about 25% w/w, about 1% to about 20% w/w, about 1% to about 15% w/w, about 1% to about 20% w/w, or about 1% to about 2.5% w/w of the one or more skin penetration enhancers.

In certain embodiments, the pharmaceutical composition comprises about 0.05% to about 2.5% w/w or about 0.05% to about 2.0% w/w of the one or more preservatives.

In certain embodiments, the pharmaceutical composition comprises about 1.5% to about 3% w/w of the one or more preservatives.

In certain embodiments, the pharmaceutical composition comprises about 2% w/w of the one or more preservatives.

In certain embodiments, the pharmaceutical composition comprises about 0.05% to about 0.2% w/w, about 0.05% to about 0.18% w/w, or about 0.05% to about 0.1% w/w of the one or more preservatives.

In certain embodiments, the pharmaceutical composition comprises about 0.5% to about 9% w/w, about 0.5% to about 4.3% w/w, or about 0.5% to about 4% w/w of the one or more gelling agents.

In certain embodiments, the pharmaceutical composition comprises about 1% to about 9% w/w, about 1% to about 4% w/w, or about 1% to about 1.75% w/w of the one or more gelling agents.

In certain embodiments, the pharmaceutical composition comprises about 1% w/w of the one or more gelling agents.

### METHODS OF THE INVENTION

The present invention provides a method for preventing or treating nausea and/or vomiting, comprising administering to a subject in need thereof a therapeutically effective amount of the pharmaceutical composition of the present invention, thereby preventing or treating the nausea and/or vomiting.

In certain embodiments, the nausea and/or vomiting are caused by cancer chemotherapy, radiation therapy, drugs (e.g., hydromorphone, acepromazine, and glycopyrrolate), gastrointestinal disturbances or infection, vestibular syndrome, and/or surgery.

In certain embodiments, the subject is a non-human primate.

In certain embodiments, the subject is a human.

In certain embodiments, the subject is a non-human subject.

In certain embodiments, the non-human subject is a canine.

In certain embodiments, the non-human subject is a feline.

In certain embodiments, the canine is a puppy.

In certain embodiments, the feline is a kitten.

In certain embodiments, the pharmaceutical composition is administered topically.

In certain embodiments, the pharmaceutical composition is administered to a pinna or an axilla.

In certain embodiments, a dosage of about 1 mg/kg to about 20 mg/kg ondansetron hydrochloride or pharmaceutically acceptable salt thereof is administered.

In certain embodiments, a dosage of about 5 mg/kg to about 20 mg/kg is administered.

In certain embodiments, a dosage of about 10 mg/kg to about 20 mg/kg is administered.

In certain embodiments, a dosage of about 5 mg/kg to about 15 mg/kg is administered.

In certain embodiments, a dosage of about 5 mg/kg to about 10 mg/kg is administered.

In certain embodiments, a dosage of about 10 mg/kg to about 15 mg/kg is administered.

In certain embodiments, a dosage of about 15 mg/kg to about 20 mg/kg is administered.

In certain embodiments, a dosage of about 6 mg/kg is administered.

In certain embodiments, a dosage of about 12 mg/kg is administered.

In certain embodiments, a dosage of about 18 mg/kg is administered.

In certain embodiments, the pharmaceutical composition is administered once daily.

In certain embodiments, the pharmaceutical composition is administered twice daily.

In certain embodiments, the pharmaceutical composition is administered about every 12 to 72 hours.

In certain embodiments, the pharmaceutical composition is administered about every 12 to 24 hours.

In certain embodiments, the pharmaceutical composition is administered about every 12 hours.

In certain embodiments, the pharmaceutical composition is administered about every 24 hours.

In certain embodiments, the pharmaceutical composition is administered about every 12 hours for 3 to 5 doses following by about every 24 hours for 3 to 5 doses.

In certain embodiments, a first dosage is administered about every 12 hours for 3 to 5 doses following by a second dosage about every 12 hours for 3 to 5 doses.

In certain embodiments, the first dosage is administered about every 12 hours for 3 to 5 doses following by the second dosage about every 12 hours for 8-12 doses.

### Administration

The formulations used in the invention may be administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the composition of the invention can be administered to a subject by any mode that delivers the compound or composition to the desired location or surface. Administering the pharmaceutical composition of the present invention may be accomplished by any means known to the skilled artisan. Routes of administration include but are not limited to oral, intravenous, intramuscular, intraperitoneal, subcutaneous, direct injection (for example, into a tumor or abscess), mucosal, inhalation, and topical.

In certain embodiments, the composition is administered systemically. In certain preferred embodiments, the composition is administered intravenously.

The pharmaceutical composition of the present invention is administered to skin, e.g. as a transdermal gel. Upon application of the transdermal gel, the active drug, i.e., ondansetron or pharmaceutically acceptable salt or hydrate thereof, is absorbed through the skin. Administering ondansetron through the transdermal route avoids hepatic first-pass metabolism. The delivery of ondansetron to the systemic circulation via the transdermal route could improve its systemic bioavailability.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing and/or dispersing agents.

The pharmaceutical compositions may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The pharmaceutical compositions may comprise suitable solvents, skin penetration enhancers, preservatives, and/or gelling agents.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990), which is incorporated herein by reference.

The compounds of the invention and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the invention contain an effective amount of a compound of the invention and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agent(s), including specifically but not limited to the compound of the invention, may be provided in particles. Particles as used herein means nanoparticles or microparticles (or in some instances larger particles) which can consist in whole or in part of the compound of the invention or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, non-erodible, biodegradable, or nonbiodegradable material or combinations thereof. The particles may be microcapsules which contain the compound of the invention in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

### Definitions

Ondansetron is a serotonin 5-HT3 receptor antagonist. The mechanism of action is to block the action of serotonin, a natural substance that may cause nausea and vomiting. It is commonly prescribed as an antiemetic to prevent nausea and vomiting caused by cancer chemotherapy, radiation therapy, and surgery. The w/w % ondansetron may be calculated based on the amount of the pharmaceutically acceptable salt, or the amount of the dihydrate of the pharmaceutically acceptable salt.

Ondansetron has the following chemical structure:

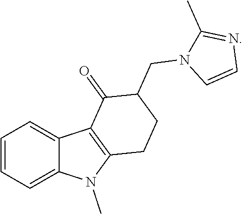 [SMILES: Cc1nccn1CC1CCc2c(c3ccccc3n2C)C1=O]

The hydrochloride dihydrate form of ondansetron has the following chemical structure:

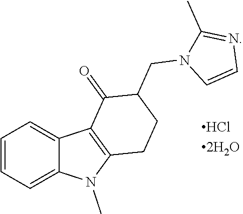 [SMILES: Cc1nccn1CC1CCc2c(c3ccccc3n2C)C1=O]

The terms “treat” and “treating” as used herein refer to performing an intervention that results in (a) preventing a condition or disease from occurring in a subject that may be at risk of developing or predisposed to having the condition or disease but has not yet been diagnosed as having it; (b) inhibiting a condition or disease, e.g., slowing or arresting its development; or (c) relieving or ameliorating a condition or disease, e.g., causing regression of the condition or disease. In one embodiment the terms “treating” and “treat” refer to performing an intervention that results in (a) inhibiting a condition or disease, e.g., slowing or arresting its development; or (b) relieving or ameliorating a condition or disease, e.g., causing regression of the condition or disease.

A “subject” or “patient” as used herein refers to a living mammal. In various embodiments, a subject or patient is a non-human mammal, including, without limitation, a mouse, rat, hamster, guinea pig, rabbit, sheep, goat, cat, dog, pig, horse, cow, or non-human primate. In certain embodiments, a subject or patient is a human. A “non-human subject” or “non-human patient” as used herein exclude humans.

“Effective amount” as used herein refers to any amount that is sufficient to achieve a desired biological effect.

“Therapeutically effective amount” as used herein refers to any amount that is sufficient to achieve a desired therapeutic effect, e.g., treating nausea.

“Active ingredient”, “therapeutically active ingredient”, “active agent”, “drug” or “drug substance” as used herein means the active ingredient of a pharmaceutical, also known as an active pharmaceutical ingredient (API).

“Amorphous” as used herein refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid-like properties occurs at a “glass transition”, typically defined as a second-order phase transition.

“Crystalline” as used herein refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically a first-order phase transition (“melting point”). In the context of the present invention, a crystalline active ingredient means an active ingredient with crystallinity of greater than 75%. In certain embodiments the crystallinity is suitably greater than 90%. In other embodiments, the crystallinity is greater than 95%. In other embodiments, the crystallinity is less than 10%, or less than 5%.

“Drug Loading” as used herein refers to the percentage of active ingredient(s) on a mass basis in the total mass of the formulation.

The term “about” refers to variations in numerical values typically encountered by one of skill in the art of respirable formulations, including variations of plus or minus 0.1%, 0.5%, 1%, 2%, 3%, 4%, 5%, 6%, 7%, 8%, 9%, or 10% of a numerical value described herein.

Throughout this specification and in the claims that follow, unless the context requires otherwise, the word “comprise”, or variations such as “comprises” or “comprising”, should be understood to imply the inclusion of a stated integer or step or group of integers or steps but not the exclusion of any other integer or step or group of integers or steps.

Unless otherwise stated, or clear from the context, numerical ranges include both the endpoints and any value between them.

### EXAMPLES

Having now described the present invention in detail, the same will be more clearly understood by reference to the following examples, which are included herewith for purposes of illustration only and are not intended to be limiting of the invention.

### Example 1: Preparation of Compositions

### Preparation Procedure No. 1

An exemplary composition (13B) was prepared as followed:
Step 1—Gel Phase
   A) Weigh required amount of glycerin (112.5 g) into a glass beaker (GB-1) and stir at 200-500 RPM for about 4 h.
   B) Weigh required amount of hydroxypropylcellulose (HPC) (7.5 g) and add slowly to step 1A contents. Continue mixing for complete polymer hydration. Note: Nasal mask must be worn while handling HPC.
Step 2—Solvent Phase
   A) Weigh and add required amounts of propylene glycol (221.2 g) and Ondansetron HCl (41.239 g) to a stainless steel vessel (SS-1) and subject to overhead mixing while heating on a water bath to 60-65° C. for about 1 h until fully dissolved. Be sure to protect from light, as API is light sensitive.
   B) Weigh and add required amounts of Transcutol V, dimethyl isosorbide, benzyl alcohol, hexylene glycol, polysorbate 80, and phenoxyethanol and add to another glass beaker (GB-2) in the following order. Subject to overhead mixing for about 2.5 h until a uniform mixture is achieved.
   Transcutol V: 285.0 0 g; Dimethyl isosorbide: 37.5 g; Benzyl alcohol: 15.0 g; Hexylene Glycol: 7.5 g; Polysorbate 80: 15 g; Phenoxyethanol: 7.5 g.
Step 3—Phase Transfer (Gel Phase Addition to Solvent Phase)
   A) Add the gel phase contents from step 1A to the step 2A contents in SS-1 followed by the addition of step 2B contents in GB-2 to SS-1 and subject to overhead mixing with a trident blade at 35-150 RPM for about 1 h until homogenous mixing of the bulk gel is achieved. Be sure to protect from light, as API is light sensitive.
   B) If needed, homogenize for 5-10 minutes at 1500-3500 RPM to achieve a uniform gel.
Step 4—Transfer and Storage
   Transfer the bulk gel with an inert scraper into a light-protected container.

The above procedure was used or adapted to prepare the compositions described in Table 1A and Table 1B.

### Preparation Procedure No. 2

The composition was prepared by the following method on a 3 kg scale using an Axomix 5 L homogenizer. Temperatures, mixing times, and mixing speeds may be adapted during scale-up of the process.
Step 1—Add propylene glycol (Part 1), benzyl alcohol, and ondansetron HCl to the axomix vessel. Rinse the API container with propylene glycol (Part 2) and transfer rinsate to the axomix vessel. (axomix jacket temperature=40-43° C., product temperature=35-40° C.)
Step 2—Mix at 500 rpm until complete dissolution of the ondansetron HCl is achieved.
Step 3—In this order add transcutol V, dimethyl isosorbide, hexylene glycol, polysorbate 80, phenoxyethanol, and glycerin to the axomix vessel. (axomix jacket temperature=22-25° C., product temperature=22-25° C., vacuum=−0.2-−0.6 bar)
Step 4—Mix at 80 rpm until a uniform clear to pale yellow solution with no undissolved particles is obtained (mixing time=NLT 10 minutes)
Step 5—Initiate homogenization (homongenization speed=2000 rpm) and mixing (mixing speed=90 rpm). Engage vacuum (−0.2-−0.6 bar). Transfer in hydroxypropyl cellulose through the vacuum port. After complete transfer close the vacuum port and homogenize for 30 minutes while mixing for NLT 2 hours. Continue mixing, if necessary, until a homogeneous bulk gel is obtained.
Step 6—Fill the drug product into aluminum tubes and seal.

TABLE 1A

| Component | Function | 3* | 3A* | 3B* | 3C* | 3D* | 3E* | 3G* | 3H* |
|---|---|---|---|---|---|---|---|---|---|
| Ondansetron HCl** | API | 5.5 | 5.5 | 5.5 | 5.5 | 5.5 | 5.5 | 5.5 | 5.5 |
| Propylene glycol | Solvent | 28.5 | 25.5 | 22.5 | 22.5 | 35.5 | 30.5 | 37.5 | 34.5 |
| Transcutol ® V | Penetration | 20 | 25 | 30 | — | 30 | 30 | 30 | 30 |
| | enhancer | | | | | | | | |
| Transcutol ® HP | Penetration | — | — | — | 30 | — | — | — | — |
| | enhancer | | | | | | | | |
| Dimethyl isosorbide | Penetration | — | — | 2 | — | — | — | — | — |
| | enhancer | | | | | | | | |
| Medium chain | Oil/ | 18 | 18 | 18 | 18 | 5 | 10 | 5 | 10 |
| triglycerides (MCT) | Penetration | | | | | | | | |
| | enhancer | | | | | | | | |
| Cyclomethicone | Oil/Skin | 2 | 2 | 2 | 2 | 2 | 2 | — | — |
| | conditioning | | | | | | | | |
| | agent | | | | | | | | |
| Glycerin | Humectant/ | 20 | 15 | 15 | 15 | 15 | 15 | 15 | 15 |
| | Solvent | | | | | | | | |
| Benzyl alcohol | Preservative | 2 | 2 | 2 | 2 | 2 | 2 | 2 | 2 |
| Hydroxypropylmethyl | Gelling | 3 | 3 | — | — | — | — | — | — |
| cellulose (HPMC) | agent | | | | | | | | |
| Hydroxypropyl | Gelling | 1 | 2 | 1 | 3 | 3 | 3 | 3 | 3 |
| cellulose (HPC) | agent | | | | | | | | |
| Polysorbate 80 | Solubiliser | — | 2 | 2 | 2 | 2 | 2 | 2 | — |

*All amounts are recited as % w/w.
**% w/w includes water content (dihydrate).

TABLE 1B

| Component | Function | 13* | 13A* | 13B* | 13C* |
|---|---|---|---|---|---|
| Ondansetron HCl** | API | 5.5 | 5.5 | 5.5 | 5.5 |
| Propylene glycol | Solvent | 42.5 | 29.5 | 29.5 | 29.5 |
| Transcutol ® V | Penetration | 30 | 33 | 38 | 38 |
| | enhancer | | | | |
| Dimethyl isosorbide | Penetration | — | 10 | 5 | 5 |
| | enhancer | | | | |
| Glycerin | Humectant/ | 15 | 15 | 15 | 15 |
| | Solvent | | | | |
| Hexylene glycol | Solvent | 1 | 1 | 1 | — |
| Benzyl alcohol | Preservative | 2 | 2 | 2 | 2 |
| Hydroxypropyl | Gelling | 1 | 1 | 1 | 3 |
| cellulose (HPC) | agent | | | | |
| Polysorbate 80 | Solubiliser | 2 | 2 | 2 | 2 |
| Phenoxyethanol | Preservative | 1 | 1 | 1 | — |

*All amounts are recited as % w/w.
**% w/w includes water content (dihydrate).

### Example 2: Visual Solubility Screen Report for Ondansetron HCl

The purpose of this study was to evaluate the solubility of Ondansetron HCl in an array of solvents/excipients to develop a stable topical formulation for transdermal application in cats and dogs. In this screen, evaluation of solubility was determined by visual examination only.

The following procedure was used to determine solubility of the API (Ondansetron HCl):
a. Label 20 mL USP Type I scintillation vials appropriately for each solvent/solvent blend that will be tested. Although API is light sensitive, clear glass vials will be used, as the study is a visual assessment only.
b. Place a stir bar in each vial. Weigh the empty vial containing stir bar and cap with inert liner. Record the tare weight.
c. Weigh approximately 3.0 g of each solvent into the designated vial.
d. Weigh approximately 30.0 mg of the API into each vial.
e. Place all closed vials on a stir plate and allow mixing for at least 30 minutes at ambient conditions.
f. Visually inspect to see if the API has completely dissolved. Record the stir time and observations.
g. If the API has dissolved, repeat steps d to f until saturation solubility is achieved or up to 10% w/w.
h. Record additional API aliquots added to the mixture. Continue stirring solutions overnight at ambient temperature to confirm solubility is maintained.
i. Record the final weight of the vial, cap, stir bar, and sample to calculate the total amount of active and/or solvent added at room temperature.
j. Heat (if applicable) all the vials in which API is not soluble to 65° C. for 1 hour and check the solubility. Allow them to cool to room temperature and check for any precipitation in the vials in which API was soluble when heated. Perform microscopy as deemed necessary.
k. Confirm the pH of buffer solutions after addition and solubilization (if applicable) of the API.
l. Record final observations and take pictures as needed to document the observations.

### Primary Solvent Solubility

Upon addition of Ondansetron HCl to each solvent, vials were subjected to stirring at room temperature at 400 RPM.

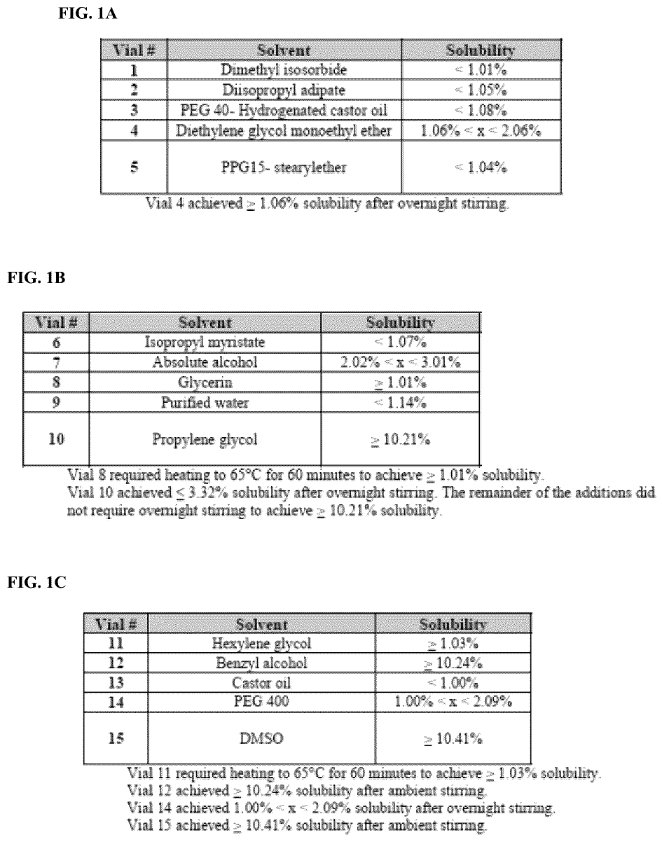

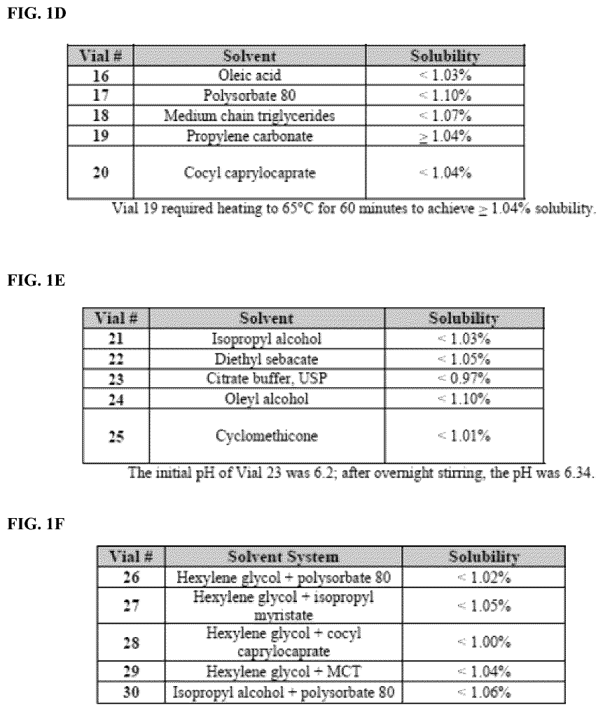

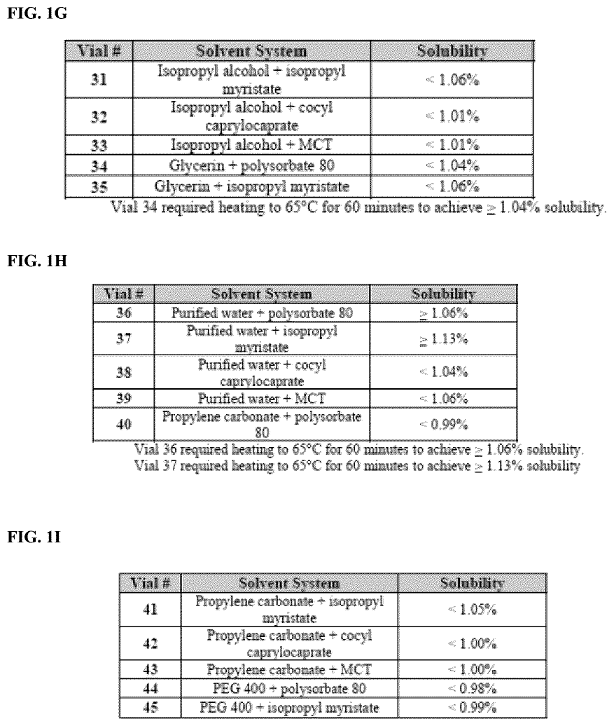

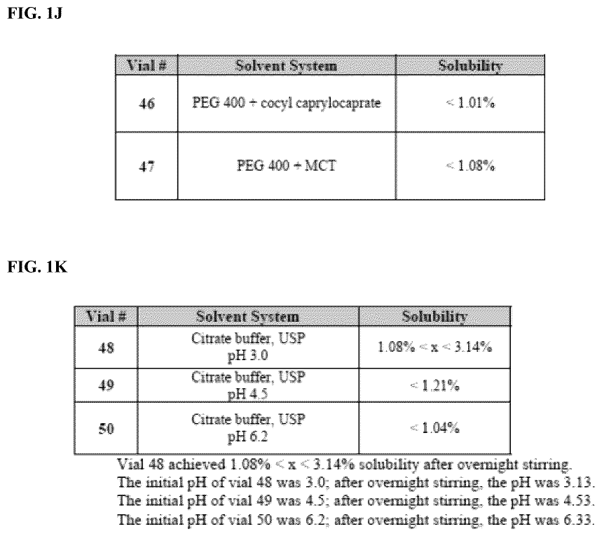

The solubility in dimethyl isosorbide, diisopropyl adipate, PEG 40-Hydrogenated castor oil, diethylene glycol monoethyl ether, or PPG15-stearylether is shown in FIG. 1A.

The solubility in isopropyl myristate, absolute alcohol, glycerin, purified water, or propylene glycol is shown in FIG. 1B.

The solubility in hexylene glycol, benzyl alcohol, castor oil, PEG 400, and DMSO is shown in FIG. 1C.

The solubility in oleic acid, polysorbate 80, medium chain triglycerides, propylene carbonate, or cocyl caprylocaprate is shown in FIG. 1D.

The solubility in isopropyl alcohol, diethyl sebacate, citrate buffer USP, oleyl alcohol, or cyclomethicone is shown in FIG. 1E.

### Secondary Solvent Solubility

For the secondary solvent solubility screen, solvents which required heating as primary systems were blended at a 1:1 ratio with other solubilizers being screened.

The solubility in hexylene glycol+polysorbate 80, hexylene glycol+isopropyl myristate, hexylene glycol+cocyl caprylocaprate, hexylene glycol+MCT, or isopropyl alcohol+polysorbate 80 is shown in FIG. 1F.

The solubility in isopropyl alcohol+isopropyl myristate, isopropyl alcohol+cocyl caprylocaprate, isopropyl alcohol+mct, glycerin+polysorbate 80, or Glycerin+isopropyl myristate is shown in FIG. 1G.

The solubility in purified water+polysorbate 80, purified water+isopropyl myristate, purified water+cocyl caprylocaprate, Purified water+MCT, or propylene carbonate+polysorbate 80 is shown in FIG. 1H.

The solubility in Propylene carbonate+isopropyl myristate, Propylene carbonate+cocyl caprylocaprate, Propylene carbonate+MCT, PEG 400+polysorbate 80, and PEG 400+isopropyl myristate is shown in FIG. 1I.

The solubility in PEG 400+cocyl caprylocaprate or PEG 400+MCT is shown in FIG. 1J.

The solubility in citrate buffer, USP pH 3.0 or PEG 400+MCT is shown in FIG. 1K.

### Results

Based on the data from the primary solvent solubility, Ondansetron HCl can be solubilized (without heating) to at least 1% w/w in the following solvents: diethylene glycol monoethyl ether, absolute alcohol, propylene glycol, benzyl alcohol, PEG 400, DMSO, and Citrate buffer USP (pH 3.0). Solubility of ≥10% was achieved with propylene glycol, benzyl alcohol, and DMSO.

### Example 3: PK Studies

Various formulations were tested in PK studies in dogs.

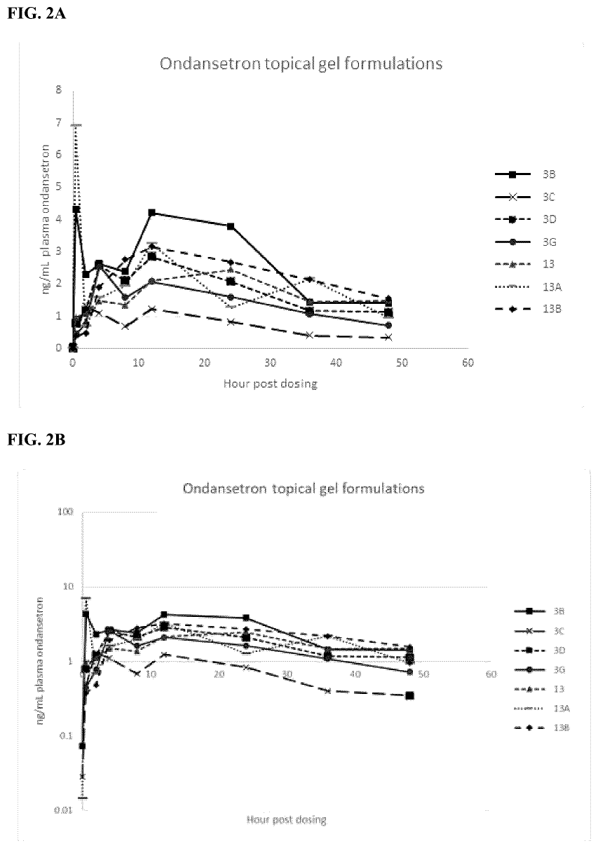

FIG. 2A shows the mean plasma concentration of various formulations for 48 h post dosing at 3 mg/kg in the pinna. FIG. 2B shows the same data on an exponential graph.

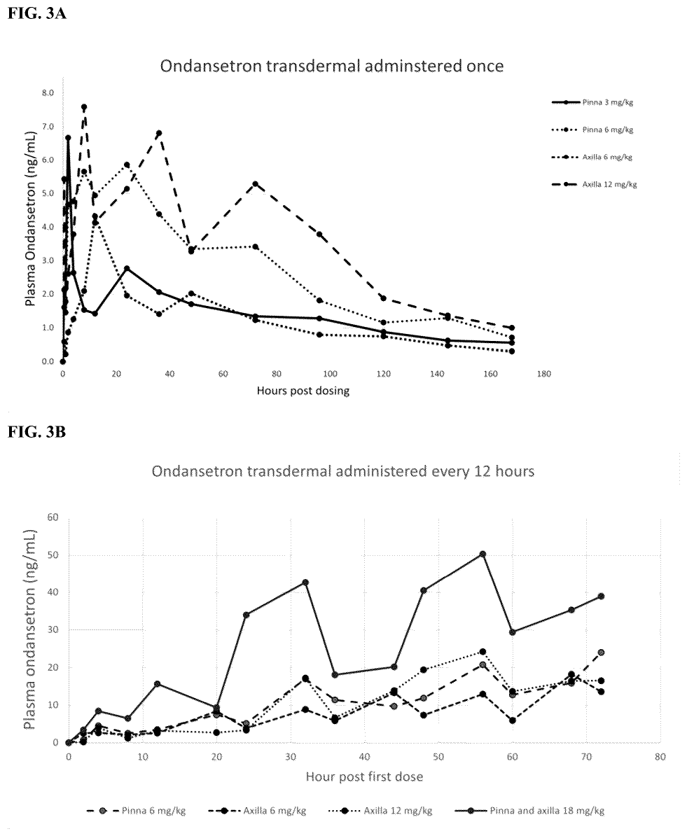

Based on the data, formulation 13B was tested further. FIG. 3A shows the mean plasma concentration of formulation 13B for about 170 h post dosing at 3 mg/kg or 6 mg/kg in the pinna and 6 mg/kg or 12 mg/kg in the axilla. FIG. 3B shows the mean plasma concentration of formulation 13B post-dosing every 12 hours for 60 hours at 6 mg/kg or 18 mg/kg in the pinna and 6 mg/kg, 12 mg/kg, or 18 mg/kg in the axilla.

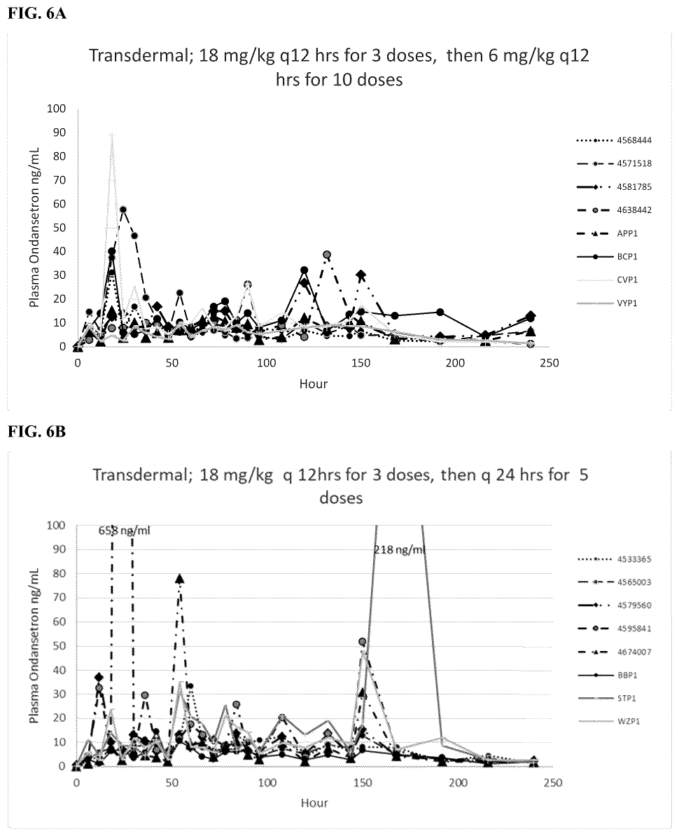

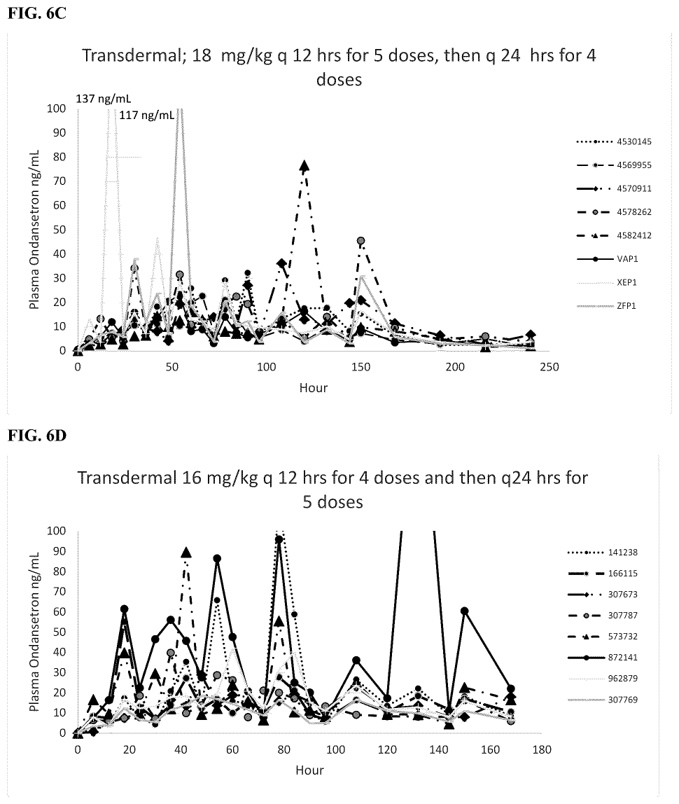

Additional dosing regimens were employed to obtain the plasma concentrations recited in in FIGS. 6A-6D. The dosing stopped at 144 hours, but PK sampling continued until 244 hrs. This data showed extended release (i.e. API still being absorbed after the last dose at 144 hrs).

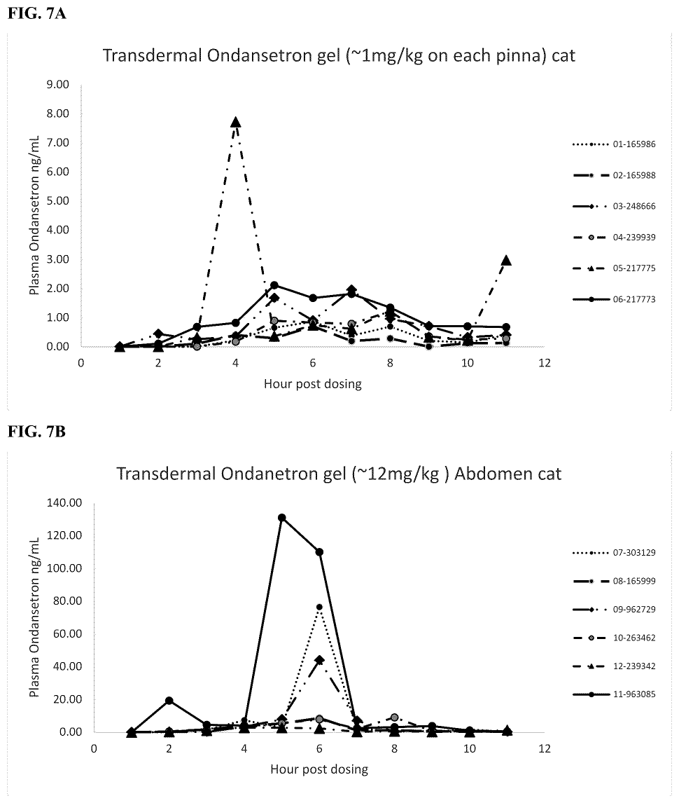

Additional dosing on the pinna or abdomen were employed to obtain the plasma concentrations in cats recited in FIGS. 7A-7B. The cats received one dose transdermally on the ears (pinnas) or the abdomen.

### Example 4: Efficacy Data

### Study Objective

This study was conducted to determine the ondansetron dose required of 13B to prevent or reduce acute vomiting and nausea induced by cisplatin when compared to a placebo control and positive control.

### Study Design

This study was conducted according to a parallel design. Forty dogs were randomly allocated to 5 groups (8 dogs per group), each of which represents a specific test/control article administration. At Time (T) 0 hour, emesis was induced chemically using the intravenous cisplatin model.

TABLE 2
Summary of test/control article administration.

| Group | No. of | Treatment | | | |
|---|---|---|---|---|---|
| No. | Dogs | Description | Route | Dose (mg/kg) | Frequency |
| 1 | 8 | Negative control | NA | 0 | Cisplatin at T = 0 |
| 2 | 8 | IVP 6 mg/kg | Topical | 6 | Treatment at |
| | | | Pinna | 3 per ear | T = −24, −12, −1 |
| 3 | 8 | IVP 12 mg/kg | Topical | 12 | |
| | | | Pinna and Axilla | 3 per ear; 3 per axilla | |
| 4 | 8 | IVP 18 mg/kg | Topical | 18 | |
| | | | Pinna and Axilla | 3 per ear; 6 per axilla | |
| 5 | 8 | Positive control | Oral | 1 | Cisplatin at T = 0 |
| | | | | | Treatment at T = −1 |

IVP: Investigational veterinary product.

Dogs was observed for 6 hours for incidents of vomiting and retching as well as signs of nausea. Vomiting was defined as expulsion of gastric contents through forceful contractions of the abdominal muscles, and retching was defined as a non-productive act of emesis. The number of episodes a dog has emesis or retches was individually tallied within each 10-min observation period. Signs of nausea included salivation, lip licking, exaggerated swallowing motions and vocalization. The number of minutes of nausea was recorded within each 10-min observation period.

### Efficacy Endpoints

Emesis and nausea were assessed from the following endpoints. 1) Primary: Total counts of vomiting episodes over a 6-hour period post cisplatin induction; Total counts of retching episodes over a 6-hour period post cisplatin induction. 2) Secondary: Total minutes of nausea over a 6-hour period post cisplatin induction

### Statistical Analysis

The study data was summarized in report tables and visually represented. Study efficacy endpoints were analyzed separately using the linear mixed model with treatment group being the fixed effect. The random effect was dog, i.e. the error term. The variance of the error term was taken as heterogeneous with respect to treatment group. The following planned comparisons were made.
Group 5 vs. Group 1
Groups 2, 3, 4 vs Group 1
Groups 2, 3, 4 vs Group 5

Effects of treatment groups were evaluated based on least squares means (LSMs), their standard errors (SEMs) and differences of LSMs. The mixed-model analysis was conducted using SAS MIXED procedure with option setting of DDFM=KR in the MODEL statement. All reported P values were two-tailed. Statistical significance was concluded at the 0.10 level, with no multiplicity adjustment. Analyses were performed using SAS software (Version 9.4, SAS Institute Inc.; Cary, NC; USA).

### Results

TABLE 3
Statistical analysis results.

| | Treatment | | Diff. (P-value†) to | | | |
|---|---|---|---|---|---|---|
| | Group | LSM +/− SEM | Neg. Ctrl | | Pos. Ctrl | |
| Emesis Episodes | Neg. Ctrl | 15.8 +/− 1.7 | — | | — | |
| (Total cnts) | IVP 6 mg/kg | 8.8 +/− 2.0 | −7.0 | (0.018) | 7.8 | (0.006) |
| | IVP 12 mg/kg | 2.7 +/− 1.8 | −13.0 | (<.001) | 1.8 | (0.387) |
| | IVP 18 mg/kg | 0.9 +/− 0.5 | −14.9 | (<.001) | −0.1 | (0.869) |
| | Pos. Ctrl | 1.0 +/− 0.5 | −14.8 | (<.001) | — | |
| Retching Episodes | Neg. Ctrl | 100.5 +/− 33.3 | — | | — | |
| (Total cnts) | IVP 6 mg/kg | 30.9 +/− 7.5 | −69.6 | (0.077) | 29.4 | (0.006) |
| | IVP 12 mg/kg | 4.4 +/− 3.1 | −96.1 | (0.023) | 2.9 | (0.396) |
| | IVP 18 mg/kg | 6.6 +/− 4.9 | −93.9 | (0.026) | 5.1 | (0.340) |
| | Pos. Ctrl | 1.5 +/− 1.0 | −99.0 | (0.021) | — | |
| Nausea Time | Neg. Ctrl | 43.3 +/− 17.7 | — | | — | |
| (Total mins) | IVP 6 mg/kg | 13.0 +/− 4.2 | −30.3 | (0.136) | 12.0 | (0.025) |
| | IVP 12 mg/kg | 2.5 +/− 1.2 | −40.8 | (0.055) | 1.5 | (0.323) |
| | IVP 18 mg/kg | 0.4 +/− 0.2 | −42.9 | (0.046) | −0.6 | (0.445) |

| | | | | |
|---|---|---|---|---|
| Pos. Ctrl | 1.0 +/− 0.8 | −42.3 | (0.048) | — |

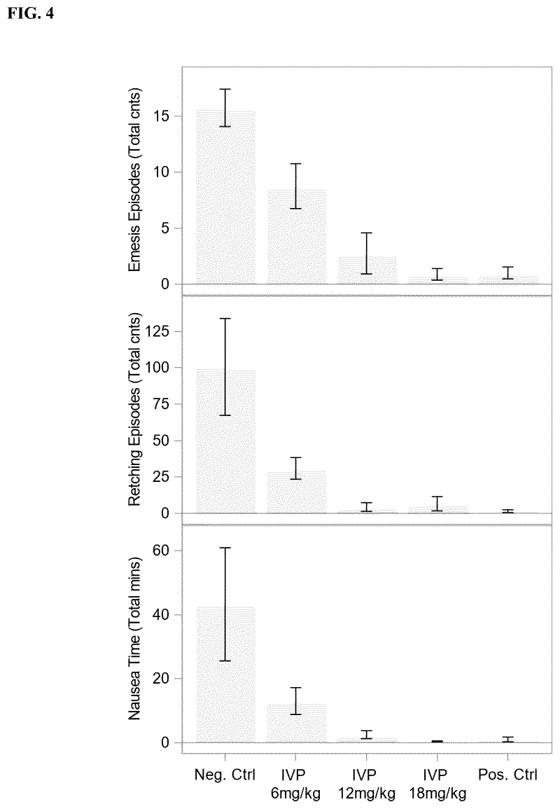

FIG. 4 presents bar charts of the statistical analysis results.

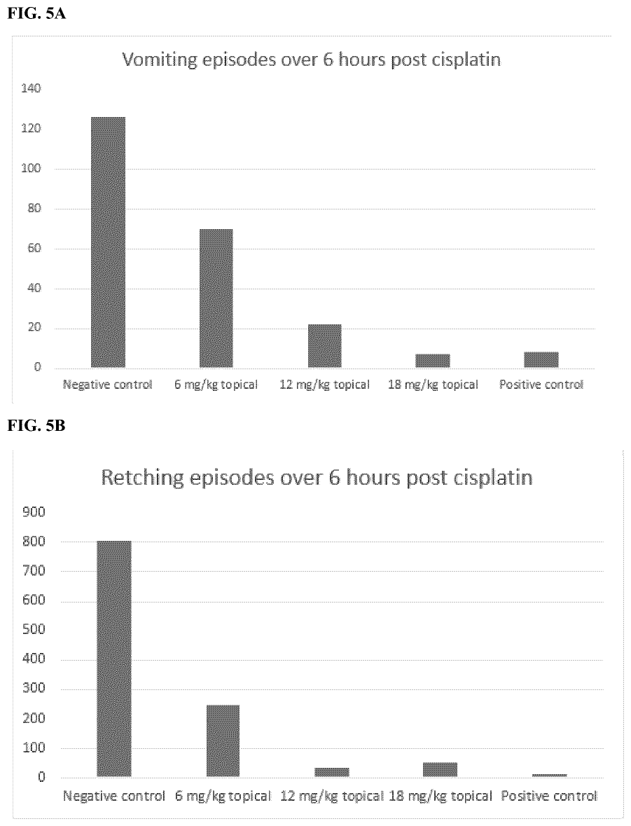

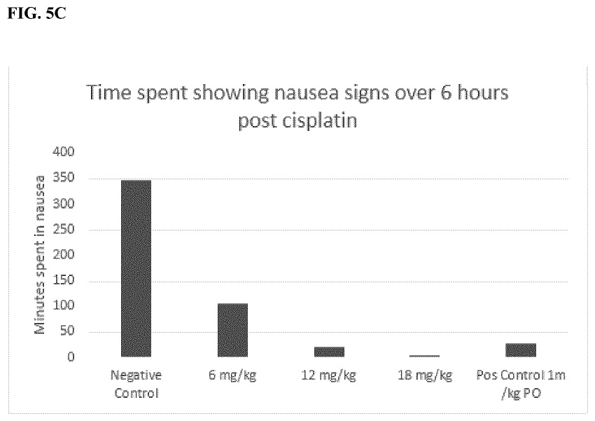

Vomiting and retching episodes as well as minutes spent showing signs of nausea are shown in FIGS. 5A-5C. The data shows the composition reduced the number of vomiting, retching episodes, and duration of nausea.

### Example 5: 200 kg-Scale Ondansetron Transdermal Gel Manufacturing

The composition was prepared by the following method on 200 kg scale using an 300 L Axomix homogenizer.
Step 1—Transfer propylene glycol and benzyl alcohol into the 300 L axomix vessel. Initiate heating to 40±2° C.
Step 2—When the contents reaches 40±2° C., transfer Ondansetron HCl into the Axomix and initiate homogenization. Maintain the phase temperature at 40±2° C. Mix at 500 RPM (450-550 RPM) until complete dissolution of the ondansetron HCl is achieved.
Step 3—Cool the contents of the vessel to 30+2° C. and add in this order DMI, glycerin, transcutol V, polysorbate 80, phenoxyethanol, hexylene glycol, and phenoxyethanol to the axomix vessel. (vacuum=−0.2-−0.5 bar)
Step 4—Mix at 70 rpm until a uniform clear to pale yellow solution with no undissolved particles is obtained (mixing time=NLT 10 minutes)
Step 5—Initiate homogenization (homongenization speed=2000 rpm) and mixing (mixing speed=70 rpm). Engage vacuum (−0.2-−0.5 bar). Transfer in hydroxypropyl cellulose through the vacuum port. After complete transfer close the vacuum port and homogenize for NLT 30 minutes while mixing for NLT 1.5 hours. Continue mixing if necessary until a homogeneous bulk gel is obtained.
Step 6—Fill the drug product into aluminum tubes and seal.

### INCORPORATION BY REFERENCE

All US patents and published US and PCT patent applications mentioned in the description above are incorporated by reference herein in their entirety.

### EQUIVALENTS

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

1. A pharmaceutical composition, comprising;
(i) about 2% to about 10% w/w ondansetron or a pharmaceutically acceptable salt thereof;
(ii) about 10% to about 50% w/w of one or more solvents;
(iii) about 10% to about 50% w/w of one or more skin penetration enhancers;
(iv) about 1% to about 5% w/w of one or more preservatives; and
(v) about 1% to about 20% w/w of one or more gelling agents.

2. The pharmaceutical composition of claim 1, wherein the one or more solvents are selected from the group consisting of propylene glycol, hexylene glycol, isopropyl alcohol, ethanol, isopropyl myristate, dimethyl sulfoxide, and water.

3. (canceled)

4. The pharmaceutical composition of claim 1, wherein the one or more skin penetration enhancers are selected from the group consisting of diethylene glycol monoethyl ether, oleyl alcohol, oleic acid, dimethyl isosorbide, cocoyl caprylocaprate, and medium chain triglyceride.

5. The pharmaceutical composition of claim 4, wherein the medium chain triglyceride comprises caproic acid, caprylic acid, capric acid, or lauric acid moieties or combinations thereof.

6. (canceled)

7. The pharmaceutical composition of claim 1, wherein the one or more preservatives are selected from the group consisting of benzyl alcohol, sorbic acid, benzoic acid, and phenoxyethanol.

8. (canceled)

9. The pharmaceutical composition of claim 1, wherein the one or more gelling agents are selected from the group consisting of hydroxypropyl cellulose, hydroxypropylmethyl cellulose, hydroxyethyl cellulose, sodium carboxymethyl cellulose, and sodium alginate.

10. (canceled)

11. The pharmaceutical composition of claim 1, comprising about 4% to about 8% w/w ondansetron or a pharmaceutically acceptable salt thereof.

12. The pharmaceutical composition of claim 11, comprising about 5% to about 6% w/w ondansetron or a pharmaceutically acceptable salt thereof.

13. (canceled)

14. The pharmaceutical composition of claim 1, comprising about 10% to about 45% w/w of the one or more solvents.

15. The pharmaceutical composition of claim 14, comprising about 25% to about 35% w/w of the one or more solvents.

16. (canceled)

17. (canceled)

18. (canceled)

19. (canceled)

20. (canceled)

21. The pharmaceutical composition of claim 1, comprising about 30% to about 50% w/w of the one or more skin penetration enhancers.

22. The pharmaceutical composition of claim 21, comprising about 35% to about 45% w/w of the one or more skin penetration enhancers.

23. (canceled)

24. (canceled)

25. (canceled)

26. (canceled)

27. The pharmaceutical composition of claim 1, comprising about 1% to about 4% w/w of the one or more preservatives.

28. (canceled)

29. (canceled)

30. (canceled)

31. (canceled)

32. The pharmaceutical composition of claim 1, comprising about 1% to about 9% w/w of the one or more gelling agents.

33. The pharmaceutical composition of claim 32, comprising about 1% to about 1.75% w/w of the one or more gelling agents.

34. (canceled)

35. (canceled)

36. The pharmaceutical composition of claim 1, comprising;
(i) about 2% to about 10% w/w ondansetron or a pharmaceutically acceptable salt thereof;
(ii) about 20% to about 50% w/w of propylene glycol;
(iii) about 20% to about 50% w/w of one or more skin penetration enhancers selected from diethylene glycol monoethyl ether, dimethyl isosorbide, and medium chain triglyceride;
(iv) about 1% to about 3% w/w of one or more preservatives selected from benzyl alcohol and phenoxyethanol; and
(v) about 1% to about 10% w/w of one or more gelling agents selected hydroxypropyl cellulose and hydroxypropylmethyl cellulose.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. The pharmaceutical composition of claim 1, comprising;
(i) about 5% to about 10% w/w ondansetron or a pharmaceutically acceptable salt thereof;
(ii) about 28% to about 32% w/w of a solvent;
(iii) about 35% to about 40% w/w of a skin penetration enhancer;
(iv) about 0.05% to about 2% w/w of a preservative; and
(v) about 0.5% to about 1% w/w of a gelling agent.

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. The pharmaceutical composition of claim 1, wherein the ondansetron or pharmaceutically acceptable salt thereof is ondansetron hydrochloride dihydrate.

63. The pharmaceutical composition of claim 62, comprising;
(i) about 6.2% w/w ondansetron hydrochloride dihydrate;
(ii) about 29.4% w/w propylene glycol;
(iii) about 38% w/w diethylene glycol monoethyl ether;
(iv) about 2% w/w benzyl alcohol; and
(v) about 1% w/w hydroxypropyl cellulose.

64. The pharmaceutical composition of claim 63, further comprising (vi) a humectant, (vi) a skin conditioning agent, and/or (vii) a solubilizer.

65. (canceled)

66. (canceled)

67. (canceled)

68. (canceled)

69. (canceled)

70. The pharmaceutical composition of claim 1, wherein the pharmaceutical composition is a gel (e.g., a transdermal gel).

71. A method for preventing or treating nausea and/or vomiting, comprising administering to a subject in need thereof a therapeutically effective amount of the pharmaceutical composition of claim 1, thereby preventing or treating the nausea and/or vomiting.

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)
